(19) United States
(12) Patent Application Publication
(10) Pub. No.: US 2026/0090571 A1
(43) Pub. Date: Apr. 2, 2026

(54) Cat Food Composition for Treating or Preventing Urolithiasis and for Reducing the Incidence of Hairballs

(71) Applicant: Blue Buffalo Enterprises, Inc., Wilton, CT (US)

(72) Inventors: Nolan Z. Frantz, Allen, KS (US); Angela M. Gajda, Franklin, TN (US); Todd Harper, Minneapolis, MN (US)

(21) Appl. No.: 19/319,905

(22) Filed: Sep. 5, 2025

Related U.S. Application Data
(63) Continuation of application No. 18/903,721, filed on Oct. 1, 2024.

(51) Int. Cl. A23K 10/30; A23K 10/22; A23K 20/142; A23K 20/24; A23K 20/26; A23K 50/40; A61K 9/00; A61K 31/198; A61K 33/06; A61K 33/42; A61K 35/60; A61K 36/55; A61P 13/12

(52) U.S. Cl. CPC A23K 10/30; A23K 10/22; A23K 20/142; A23K 20/24; A23K 20/26; A23K 50/40; A61K 9/0056; A61K 31/198; A61K 33/06; A61K 33/42; A61K 35/60; A61K 36/55; A61P 13/12

(57) ABSTRACT

A cat food composition comprises ingredients that work to manage hairball formation as well as urine pH and urine saturation levels to treat or prevent urolithiasis. In some embodiments, a wet cat food composition comprises flaxseed, fish oil, calcium sulfate, methionine, sodium acid pyrophosphate (SAPP) and a source of dietary fiber. In some embodiments, a dry cat food composition comprises fish oil, methionine, taurine, potassium sulfate and a source of dietary fiber. Flaxseed and/or fish oil provide omega-3 fatty acids to minimize urolith formation. Calcium sulfate, potassium sulfate, taurine, methionine and/or SAPP help achieve an ideal urine pH between 6.15 and 6.85. The source of dietary fiber helps reduce the incidence of hairballs. In some embodiments, the cat food composition is configured to establish an average RSS struvite value less than or equal to 1.8, and/or an average RSS oxalate less than or equal to 6.0.

### CROSS-REFERENCE TO RELATED APPLICATIONS

This application represents a continuation-in-part of U.S. patent application Ser. No. 18/903,721 filed Oct. 1, 2024 entitled “Cat Food Composition for Treating or Preventing Urolithiasis”, pending. The entire content of this application is incorporated herein by reference.

### FIELD OF THE INVENTION

The invention generally pertains to pet food compositions and, more particularly, to cat food compositions formulated to treat or prevent urolithiasis and to reduce the incidence of hairballs. The invention also pertains to methods of making pet food compositions and, more particularly, to methods of making cat food compositions formulated to treat or prevent urolithiasis and to reduce the incidence of hairballs.

### BACKGROUND OF THE INVENTION

Feline lower urinary tract disease caused by urinary stones, also called uroliths, is a common ailment in cats. Uroliths cause irritation or blockage and are most often made of struvite (magnesium ammonium phosphate) or calcium oxalate, via a complex biochemical process which includes, e.g., urine supersaturation and crystal growth. Also, felines groom themselves by licking their fur, often resulting in regurgitation of hairballs, which many cat owners wish to avoid. A therapeutic or preventative diet for managing mineral levels and other factors affecting urolith formation as well as managing hairballs (e.g., preventing hairball formation) is needed for cat owners and veterinarians to manage the health and wellbeing of their cats.

### SUMMARY OF THE INVENTION

A cat food composition in accordance with the present invention comprises ingredients that work to manage urine pH and urine saturation levels and to prevent hairball formation. The cat food composition of the present invention offers a single diet that can manage the needs of felines having or potentially having issues with both uroliths and hairballs, providing a healthy urinary tract while also reducing the incidence of hairballs for the cat. In some embodiments, the cat food composition is a wet cat food composition comprising flaxseed, fish oil, calcium sulfate, methionine, sodium acid pyrophosphate (SAPP) and a source of dietary fiber. In some embodiments, the wet cat food composition further comprises taurine. In some embodiments, the cat food composition is a dry cat food composition comprising fish oil, methionine, taurine, potassium sulfate and a source of dietary fiber.

Flaxseed (e.g., flaxseed meal) and/or fish oil are included in the cat food composition of the present invention to provide omega-3 fatty acids to minimize urolith formation. In some embodiments, flaxseed (e.g., flaxseed meal) is present in the cat food composition in an amount of about 0.1% to about 10% (e.g., about 0.5% to about 5% or about 0.25% to about 2.5%). In some embodiments, fish oil (e.g., Menhaden fish oil) is present in the cat food composition in an amount of about 0.1% to about 10% (e.g., about 0.5% to about 5% or about 0.25% to about 2.5%).

Calcium sulfate, potassium sulfate, taurine, methionine (e.g., DL-methionine) and/or SAPP are included in the cat food composition of the present invention to help achieve an ideal urine pH (e.g., a pH between 6.15 and 6.85). In some embodiments, the cat food composition of the present invention includes calcium sulfate in an amount of about 0.04% to about 4% (e.g., about 0.2% to about 2% or about 0.1% to about 1%), potassium sulfate in an amount of about 0.1% to about 0.6%, taurine in an amount of 0.05% to 0.3% or of about 0.1% to about 1.0%, methionine in an amount of about 0.002% to about 0.2% (e.g., about 0.01% to about 0.1% or about 0.005% to about 0.05%), and/or SAPP in an amount of about 0.015% to about 1.5% (e.g., about 0.075% to about 0.75% or about 0.045% to about 0.45%).

A source of dietary fiber (e.g., a source of insoluble fiber) is included in the cat food composition of the present invention to reduce the incidence of hairballs (e.g., by preventing the formation of hairballs). In some embodiments, the source of dietary fiber includes cellulose (e.g., isolated cellulose fiber, cellulose powder), Miscanthus grass, beet pulp, or a combination thereof. In some embodiments, the cat food composition of the present invention includes the source of dietary fiber in an amount of 0.75% to 2.0% (e.g., 1% to 1.5%) or in an amount of about 4% to about 6% (e.g., 4% to 6%).

In some embodiments, the cat food composition is configured to establish a urine pH between 6.15 and 6.85 (e.g., between 6.15 and 6.56 or between 6.2 and 6.4 for the wet cat food embodiments, between 6.31 and 6.85 for the dry cat food embodiments). This pH range is ideal to prevent the formation of struvite and/or calcium oxalate crystals. In some embodiments, the cat food composition has a base excess within the range of about-80 to about-100 (e.g., for the wet cat food embodiments) or about-50 to about-200 (e.g., for the dry cat food embodiments). In some embodiments, the cat food composition has a calcium to phosphorous ratio in the range of 1 to 1.5. In some embodiments, the cat food composition is configured to establish an average RSS struvite value less than or equal to 1.8, and/or an average RSS oxalate less than or equal to 6.0.

A method of making a cat food composition for treating or preventing urolithiasis and preventing the incidence of hairballs in accordance with the present invention comprises combining flaxseed, fish oil, calcium sulfate, methionine, sodium acid pyrophosphate and a source of dietary fiber, or comprises combining fish oil, methionine, taurine, potassium sulfate and a source of dietary fiber. In some embodiments, combining includes combining flaxseed in an amount of about 0.1% to about 10% (e.g., about 0.5% to about 5% or about 0.25% to about 2.5%) by weight of the cat food composition, fish oil in an amount of about 0.1% to about 10% (e.g., about 0.5% to about 5% or about 0.25% to about 2.5%) by weight of the cat food composition, calcium sulfate in an amount of about 0.04% to about 4% (e.g., about 0.2% to about 2% or about 0.1% to about 1%) by weight of the cat food composition, methionine in an amount of about 0.002% to about 0.2% (e.g., about 0.01% to about 0.1% or about 0.005% to about 0.05%) by weight of the cat food composition, sodium acid pyrophosphate in an amount of about 0.015% to about 1.5% (e.g., about 0.075% to about 0.75% or about 0.045% to about 0.45%) by weight of the cat food composition and a source of dietary fiber in an amount of 0.75% to 2.0% (e.g., 1% to 1.5%) by weight of the cat food composition. In some embodiments, combining includes combining fish oil in an amount of about 0.1% to about 10% (e.g., about 0.5% to about 5% or about 0.25% to about 2.5%) by weight of the cat food composition, methionine in an amount of about 0.002% to about 0.2% (e.g., about 0.01% to about 0.1% or about 0.005% to about 0.05%) by weight of the cat food composition, taurine in an amount of about 0.1% to about 0.6% by weight of the cat food composition, potassium sulfate in an amount of about 0.1% to about 1.0% by weight of the cat food composition and a source of dietary fiber in an amount of about 4% to about 6% (e.g., 4% to 6%) by weight of the cat food composition.

### DETAILED DESCRIPTION OF EMBODIMENTS

Initially, it should be noted that the embodiments of the present invention described below are not intended to be exhaustive or limit the invention to the precise forms disclosed in the following detailed description. Rather, a purpose of the embodiments chosen and described is so that the appreciation and understanding by others skilled in the art of the principles and practices of the present invention can be facilitated.

Throughout the present description, unless otherwise specified, the concentrations expressed as percentages always refer to the weight/weight (w/w) percentage, i.e., grams of a given component per 100 g of composition, i.e., wt % or percent by weight. The term “about” refers to a deviation of up to plus/minus 10%, preferably plus/minus 5%.

The level of urinary stone formation in a cat's urinary system is dependent upon many factors including but not limited to urine mineral content, urine pH and the presence of inhibitors and promotors (e.g., magnesium cations) of stone formation. For example, struvite is more likely to form as urine pH increases while calcium oxalate is more likely to form as urine pH decreases. The presence of struvite or calcium oxalate crystals/stones is a common cause of feline lower urinary tract disease. As another concern for cat owners, hair ingested by cats during grooming can conglomerate into hairballs in the stomach or intestine. While hairballs generally do not result in severe health problems, severe cases can be associated with abdominal pain and anorexia, and treatment can require surgery or endoscopy. The present inventors have developed a cat food composition with an added source of dietary fiber to manage a cat's hairballs and with precise mineral levels and added polyunsaturated fatty acid levels to manage the cat's urine pH and urine saturation levels so as to control or even reverse the formation of struvite and calcium oxalate crystals/stones. The cat food composition may meet the requirements for the Association of American Feed Control Officials (AAFCO) “natural” designation (e.g., not containing any additives or processing aids that are chemically synthetic except in amounts as might occur in good manufacturing practices) and contains no added corn, wheat, or soybean ingredients and no artificial flavors or colorants. The cat food composition may be considered a natural food for cats with added vitamins and minerals. The cat food may also not contain any chicken by-product meal.

In some embodiments, the cat food composition is a wet cat food composition comprising flaxseed, fish oil, calcium sulfate, methionine, sodium acid pyrophosphate and a source of dietary fiber. In some embodiments, the wet cat food composition further comprises taurine. The wet cat food composition of the present invention can be a semi-moist cat food composition (i.e., having a total moisture content of from 10% to 50%, by weight), and/or a moist cat food composition (i.e., having a total moisture content of greater than 50%, by weight, e.g., about 76% moisture). In some embodiments, the cat food composition is a dry cat food composition comprising fish oil, methionine, taurine, potassium sulfate and a source of dietary fiber. The dry cat food composition of the present invention can be a kibble cat food composition (i.e., having a total moisture content of from 5% to 12%, by weight). The cat food composition can further include ingredients such as water; animal protein such as beef, chicken or fish; farinaceous matter such as brown rice flour or potato; plant protein such as pea protein; chicken broth; guar gum; taurine; potassium chloride; potassium sulfate; choline chloride; vitamin E supplement; salt; and/or fruits such as blueberries or cranberries.

Flaxseed (e.g., flaxseed meal) and/or fish oil are included in the cat food composition of the present invention to provide omega-3 fatty acids such as eicosapentaenoic acid (EPA) and docosahexaenoic acid (DHA). Polyunsaturated fatty acids such as those in flaxseed and fish oil are used to minimize urolith formation. In some embodiments, flaxseed (e.g., flaxseed meal) is present in the cat food composition in an amount of about 0.1% to about 10% (e.g., about 0.5% to about 5% or about 0.25% to about 2.5%). In some embodiments, fish oil (e.g., Menhaden fish oil) is present in the cat food composition in an amount of about 0.1% to about 10% (e.g., about 0.5% to about 5% or about 0.25% to about 2.5%).

Calcium sulfate, potassium sulfate, taurine, methionine (e.g., DL-methionine) and/or SAPP are included in the cat food composition of the present invention to help achieve an ideal urine pH (e.g., a pH between 6.15 and 6.85). In particular, calcium sulfate, potassium sulfate, taurine, or methionine are included as sources of sulfur to help achieve an ideal urine pH while sodium acid pyrophosphate is added as a mineral source of phosphorous to help achieve an ideal urine pH. In some embodiments, the cat food composition of the present invention includes calcium sulfate in an amount of about 0.04% to about 4% (e.g., about 0.2% to about 2% or about 0.1% to about 1%), potassium sulfate in an amount of about 0.1% to about 0.6%, taurine in an amount of about 0.1% to about 1.0%, methionine in an of about 0.002% to about 0.2% (e.g., about 0.01% to about 0.1% or about 0.005% to about 0.05%), and/or SAPP in an amount of about 0.015% to about 1.5% (e.g., about 0.075% to about 0.75% or about 0.045% to about 0.45%).

A source of dietary fiber (e.g., a source of insoluble fiber) is included in the cat food composition of the present invention to reduce the incidence of hairballs (e.g., by preventing the formation of hairballs). In some embodiments, the source of dietary fiber includes cellulose (e.g., isolated cellulose fiber, cellulose powder), Miscanthus grass, beet pulp, or a combination thereof. In some embodiments, the source of dietary fiber consists of cellulose (e.g., isolated cellulose fiber or cellulose powder), Miscanthus grass or beet pulp. In some embodiments, the cat food composition of the present invention includes the source of dietary fiber in an amount of 0.75% to 2.0% (e.g., 1% to 1.5%) or in an amount of about 4% to about 6% (e.g., 4% to 6%). Amounts of the source of dietary fiber greater than about 6% of the cat food composition can yield a composition that is less palatable.

Controlled levels of added magnesium help prevent the formation of struvite crystals while controlled levels of added calcium help prevent the formation of calcium oxalate crystals. In some embodiments, the cat food composition does not include an added magnesium supplement ingredient (i.e., does not include magnesium outside of magnesium naturally occurring in meat or plant sources ingredients in the cat food composition) so as not to promote stone formation.

In some embodiments, the cat food composition is configured to establish a urine pH between 6.15 and 6.85 (e.g., between 6.15 and 6.56 or between 6.2 and 6.4 for the wet cat food embodiments, between 6.31 and 6.85 for the dry cat food embodiments). This pH range is ideal to prevent the formation of struvite and/or calcium oxalate crystals. In some embodiments, the cat food composition has a base excess within the range of about-80 to about-100 (e.g., for the wet cat food embodiments) or about-50 to about-200 (e.g., for the dry cat food embodiments). The “base excess” (BE) of the cat food composition is an indication of the balance of its component cations and anions and can correlate with the cat's urine pH. BE can be calculated from the compounds in the cat food composition as detailed in Jeremias et al. “Predictive formulas for food base excess and urine pH estimations of cats.” Animal feed science and technology 182, no. 1-4 (2013): 82-92, which is incorporated herein by reference.

Another important aspect of the cat food composition is its calcium (Ca) to phosphorous (P) ratio. In some embodiments, the cat food composition has a calcium to phosphorous ratio in the range of 1 to 1.5 (i.e., Ca:P of from 1:1 to 1.5:1).

Relative supersaturation (RSS) is a measurement used to assess the risk of crystal formation in urine and is based upon urine pH and urinary concentration of select solutes (e.g., struvite or calcium oxalate). An RSS value of less than 1.0 for struvite and less than 5.0 for calcium oxalate is known to result in crystal dissolution. In some embodiments, the cat food composition is configured to establish an average RSS struvite value less than or equal to 1.8, and/or an average RSS oxalate less than or equal to 6.0. See Example 2 below.

The present invention is also directed to a method of making a cat food composition for treating or preventing urolithiasis and preventing the incidence of hairballs. The method comprises combining flaxseed, fish oil, calcium sulfate, methionine, sodium acid pyrophosphate and a source of dietary fiber (e.g., to form a wet cat food composition) or comprises combining fish oil, methionine, taurine, potassium sulfate and a source of dietary fiber (e.g., to form a dry cat food composition). In some embodiments, the step of combining includes combining flaxseed in an amount of about 0.1% to about 10% (e.g., about 0.5% to about 5% or about 0.25% to about 2.5%) by weight of the cat food composition, fish oil in an amount of about 0.1% to about 10% (e.g., about 0.5% to about 5% or about 0.25% to about 2.5%) by weight of the cat food composition, calcium sulfate in an amount of about 0.04% to about 4% (e.g., about 0.2% to about 2% or about 0.1% to about 1%) by weight of the cat food composition, methionine in an amount of about 0.002% to about 0.2% (e.g., about 0.01% to about 0.1% or about 0.005% to about 0.05%) by weight of the cat food composition, sodium acid pyrophosphate in an amount of about 0.015% to about 1.5% (e.g., about 0.075% to about 0.75% or about 0.045% to about 0.45%) by weight of the cat food composition and source of dietary fiber in an amount of 0.75% to 2.0% (e.g., 1% to 1.5%) by weight of the cat food composition. In some embodiments, the step of combining includes combining fish oil in an amount of about 0.1% to about 10% (e.g., about 0.5% to about 5% or about 0.25% to about 2.5%) by weight of the cat food composition, methionine in an amount of about 0.002% to about 0.2% (e.g., about 0.01% to about 0.1% or about 0.005% to about 0.05%) by weight of the cat food composition, taurine in an amount of about 0.1% to about 0.6% by weight of the cat food composition, potassium sulfate in an amount of about 0.1% to about 1.0% by weight of the cat food composition and a source of dietary fiber in an amount of about 4% to about 6% (e.g., 4% to 6%) by weight of the cat food composition.

In some embodiments, the method further comprises making a cat food composition configured to establish a urine pH between 6.15 and 6.85 (e.g., between 6.15 and 6.56 or between 6.2 and 6.4 for the wet cat food embodiments, between 6.31 and 6.85 for the dry cat food embodiments). In some embodiments, the method further comprises making a cat food composition having a base excess within the range of about-80 to about-100 (e.g., for the wet cat food embodiments) or about-50 to about-200 (e.g., for the dry cat food embodiments). In some embodiments, the method further comprises making a cat food composition having a calcium to phosphorous ratio in the range of 1 to 1.5. In some embodiments, the method further comprises making a cat food composition configured to establish an average RSS struvite value less than or equal to 1.8, and/or an average RSS oxalate less than or equal to 6.0.

### Examples

Example 1—Product Formulation. Table 1 illustrates select ingredients and associated weight percentages for exemplary cat food compositions in accordance with the present invention. Table 2 illustrates select nutrient content of the exemplary cat food compositions. The Concept5 Formulation System from Creative Formulation Concepts (CFC) was used to compute nutrient values, ratios and base excess. The exemplary wet cat food composition was computed to have a base excess of −100.47 and a Ca to P ratio of 1.07.

TABLE 1

| | Exemplary Wet Cat Food | Exemplary Dry Cat Food |
|---|---|---|
| | Percentage of product by | Percentage of product by |
| Ingredient | weight (%) | weight (%) |
| Water | 10-50 | 5-12 |
| Flaxseed meal | 0.5-5.0 | NA |
| Menhaden fish oil | 0.25-2.5 | 0.1-2.0 |
| Calcium sulfate | 0.2-2.0 | NA |
| SAPP | 0.01-0.1 | NA |
| Methionine | 0.075-0.75 | 0.1-0.8 |
| Cellulose | 0.75-2.0 | 3.6-6.6 |
| Taurine | 0.05-0.3 | 0.1-0.6 |
| Potassium Sulfate | NA | 0.1-1.0 |

TABLE 2

| | Exemplary Wet Cat Food | Exemplary Dry Cat Food |
|---|---|---|
| | Percentage of product by | Percentage of product by |
| Nutrient | weight (%) | weight (%) |
| Calcium | 0.30-0.66 | 0.60-1.00 |
| Phosphorous | 0.30-0.51 | 0.50-0.80 |
| Sodium | 0.10-0.30 | 0.40-0.80 |
| Magnesium | 0.02-0.04 | 0.04-0.10 |
| Sulfur | 0.10-0.30 | 0.45-0.80 |
| Potassium | 0.20-0.40 | 0.55-0.90 |

Example 2—Testing. A 42-day Relative Supersaturation (RSS) feeding test was performed on a panel of cats to validate the performance of the above-mentioned exemplary cat food compositions. A test diet was offered twice a day. A urine sample was collected for each of the cats on days 10, 20, 30, and 40 of the feeding test and tested for pH, struvite RSS and calcium oxalate RSS. The target RSS struvite value was an average of less than or equal to 1.8 and an upper 95% confidence level equal to or less than 2.5. The target RSS calcium oxalate value was an average of less than or equal to 6.0 with an upper 95% confidence level of less than or equal to 12.0. The average urine pH over all days tested was 6.32, with an average of 0.26 for struvite relative supersaturation and 1.28 for calcium oxalate relative supersaturation.

1. A cat food composition, the composition comprising:
flaxseed;
fish oil;
calcium sulfate;
methionine;
sodium acid pyrophosphate; and
a source of dietary fiber.

2. The cat food composition of claim 1, wherein the cat food composition is a wet cat food composition.

3. The cat food composition of claim 1, wherein flaxseed is present in an amount of about 0.1% to about 10% by weight of the cat food composition, fish oil is present in an amount of about 0.1% to about 10% by weight of the cat food composition, calcium sulfate is present in an amount of about 0.04% to about 4% by weight of the cat food composition, methionine is present in an amount of about 0.002% to about 0.2% by weight of the cat food composition, sodium acid pyrophosphate is present in an amount of about 0.015% to about 1.5% by weight of the cat food composition and the source of dietary fiber is present in an amount of 0.75% to 2.0% by weight of the cat food composition.

4. The cat food composition of claim 1, wherein the cat food composition is configured to establish a urine pH between 6.15 and 6.56.

5. The cat food composition of claim 1, wherein the cat food composition has a base excess within the range of about-80 to about-100.

6. The cat food composition of claim 1, wherein the cat food composition has a calcium to phosphorous ratio in the range of 1 to 1.5.

7. The cat food composition of claim 1, wherein the cat food composition is configured to establish an average RSS struvite value less than or equal to 1.8, and/or an average RSS oxalate less than or equal to 6.0.

8. The cat food composition of claim 1, wherein the source of dietary fiber includes cellulose, Miscanthus grass, beet pulp, or a combination thereof.

9. A cat food composition, the composition comprising:
fish oil;
potassium sulfate;
methionine;
taurine; and
a source of dietary fiber.

10. The cat food composition of claim 9, wherein the cat food composition is a dry cat food composition.

11. The cat food composition of claim 9, wherein fish oil is present in an amount of about 0.1% to about 1.0% by weight of the cat food composition, potassium sulfate is present in an amount of about 0.1% to about 0.6% by weight of the cat food composition, methionine is present in an amount of about 0.002% to about 0.2% by weight of the cat food composition, taurine is present in an amount of about 0.1% to about 1.0% by weight of the cat food composition and the source of dietary fiber is present in an amount of about 4% to about 6% of the cat food composition.

12. The cat food composition of claim 9, wherein the cat food composition is configured to establish a urine pH between 6.31 and 6.85.

13. The cat food composition of claim 9, wherein the cat food composition has a base excess within the range of about-50 to about-200.

14. The cat food composition of claim 9, wherein the cat food composition has a calcium to phosphorous ratio in the range of 1 to 1.5.

15. The cat food composition of claim 9, wherein the cat food composition is configured to establish an average RSS struvite value less than or equal to 1.8, and/or an average RSS oxalate less than or equal to 6.0.

16. The cat food composition of claim 9, wherein the source of dietary fiber includes cellulose, Miscanthus grass, beet pulp, or a combination thereof.

17. A method of making a cat food composition, the method comprising combining flaxseed,
fish oil,
calcium sulfate,
methionine,
sodium acid pyrophosphate and
a source of dietary fiber;
or combining
fish oil,
potassium sulfate,
methionine,
taurine and
a source of dietary fiber.

18. The method of claim 17, wherein combining includes combining flaxseed in an amount of about 0.1% to about 10% by weight of the cat food composition, fish oil in an amount of about 0.1% to about 10% by weight of the cat food composition, calcium sulfate in an amount of about 0.04% to about 4% by weight of the cat food composition, methionine in an amount of about 0.002% to about 0.2% by weight of the cat food composition, sodium acid pyrophosphate in an amount of about 0.015% to about 1.5% by weight of the cat food composition and the source of dietary fiber in an amount of 0.75% to 2.0% by weight of the cat food composition.

19. The method of claim 17, wherein combining includes combining fish oil in an amount of about 0.1% to about 10% by weight of the cat food composition, methionine in an amount of about 0.002% to about 0.2% by weight of the cat food composition, taurine in an amount of about 0.1% to about 0.6% by weight of the cat food composition, potassium sulfate in an amount of about 0.1% to about 1.0% by weight of the cat food composition and the source of dietary fiber in an amount of about 4% to about 6% by weight of the cat food composition.

20. The method of claim 17, wherein the source of dietary fiber includes cellulose, Miscanthus grass, beet pulp, or a combination thereof.
